(19) United States
(12) Patent Application Publication
(10) Pub. No.: US 2015/0197791 A1
(43) Pub. Date: Jul. 16, 2015

(54) ULTRA-RAPID AND SENSITIVE DNA DETECTION USING DNAZYME AND ON-CHIP ISOTACHOPHORESIS

(71) Applicant: KONICA MINOLTA LABORATORY U.S.A., INC., San Mateo, CA (US)

(72) Inventor: Noriaki YAMAMOTO, Foster City, CA (US)

(73) Assignee: KONICA MINOLTA LABORATORY U.S.A., INC., San Mateo, CA (US)

(21) Appl. No.: 14/590,482

(22) Filed: Jan. 6, 2015

Related U.S. Application Data
(60) Provisional application No. 61/927,237, filed on Jan. 14, 2014.

(51) Int. Cl. C12Q 1/68

(52) U.S. Cl. CPC C12Q 1/6818

(57) ABSTRACT

A DNA detection method combines DNAzyme reactions and on-chip isotachophoresis (ITP). A mixture of sample containing a target DNA and a DNAzyme sensor which is either (1) a catalytic molecular beacon or (2) a binary DNAzyme and a probe is loaded into a trailing electrolyte (TE) reservoir of a microfluidic chip. In the presence of the target DNA, the catalytic molecular beacon or the probe is cleaved to generate a fluorescent fragment. Enhanced DNAzyme reaction occurs at the TE-to-LE interface. Fluorescent signal from cleaved catalytic molecular beacon or probe is detected either at the location where DNAzyme reaction occurs or at a separate location. In the latter case, the microfluidic chip has a separation region containing a capture gel or a sieving matrix which allows the fluorescent fragment to pass through but captures or traps the uncleaved catalytic molecular beacon or probe.

### BACKGROUND OF THE INVENTION

1. Field of the Invention

This invention relates to DNA detection, and in particular, it relates to a DNA detection method that utilizes DNAzyme reactions and on-chip isotachophoresis (ITP).

2. Description of Related Art

Sensitive DNA detection techniques based on enzymatic amplification such as polymerase chain reaction (PCR) are widely used in diagnosis. However, they are time-consuming, complex (low robustness) and expensive.

Isotachophoresis (ITP) is an electrophoresis technique that uses two buffers including a high mobility leading electrolyte (LE) and a low-mobility trailing electrolyte (TE). In peak-mode ITP, sample species bracketed by the LE and TE focus into a narrow TE-to-LE interface. Due to the high concentration of sample species in a small volume at the interface, high efficiency (rapid) molecular-molecular interaction can occur.

An ultra-rapid nucleic acid detection technology using ITP is described in Rapid Detection of Urinary Tract Infections Using Isotachophoresis and Molecular Beacons, M. Bercovici et al., Analytical Chemistry 2011, 83, 4110-4117 (“Bercovici et al. Analytical Chemistry 2011”). FIG. 1 of this article, reproduced as FIG. 1 of the instant disclosure, shows the principle of detection. The article describes: “FIG. 1a schematically presents the principles of the assay. ITP uses a discontinuous buffer system consisting of LE and TE, which are typically chosen to have respectively higher and lower electrophoretic mobility than the analytes of interest. Both sample and molecular beacons are initially mixed with the TE. When an electric field is applied, all species with mobility higher than that of the TE electromigrate into the channel. Other species (including ones with lower mobility, neutral or positively charged) remain in or near the sample reservoir. Focusing occurs within an electric field gradient at interface between the LE and TE, as sample ions cannot overspeed the LE zone but overspeed TE ions.” (Id., p. 4111, left column.) “FIG. 1. (a) Schematic showing simultaneous isotachophoretic extraction, focusing, hybridization (with molecular beacons), and detection of 16S rRNA bound to a molecular beacon. Hybridization of the molecular beacon to 16S rRNA causes a spatial separation of its fluorophore and quencher pair resulting in a strong and sequence-specific increase in fluorescent signal. (b) Raw experimental image showing fluorescence intensity of molecular beacons hybridized to synthetic oligonucleotides using ITP. (c) Detection of oligonucleotides having the same sequence as the target segment of 16S rRNA. Each curve presents the fluorescence intensity in time, as recorded by a point detector at a fixed location in the channel (curves are shifted in time for convenient visualization). 100 pM of molecular beacons and varying concentrations of targets were mixed in the trailing electrolyte reservoir. The total migration (and hybridization) time from the on-chip reservoir to the detector was less than a minute.” (Id., p. 4111, right column.) A setup for the on-chip ITP assay using a microfluidic chip is shown in FIGS. 2A and 2B of the instant disclosure, reproduced from FIGS. 2 and 3(a) of the above article.

Another article that describes nucleic acid detection using ITP is Integration of On-Chip Isotachophoresis and Functionalized Hydrogels for Enhanced-Sensitivity Nucleic Acid Detection, G. Garcia-Schwarz et al., Analytical Chemistry 2012; 84(15):6366-9, Aug. 7, 2012 (“Garcia-Schwarz et al. Analytical Chemistry 2012”). This article describes “an on-chip electrokinetic assay to perform high-sensitivity nucleic acid (NA) detection. This assay integrates electrokinetic sample focusing using isotachophoresis (ITP) with a background signal-removal strategy that employs photopatterened, DNA-functionalized hydrogels. In this multistage assay, ITP first enhances hybridization kinetics between target NAs and end-labeled complementary reporters. After enhanced hybridization, migration through a DNA-functionalized hydrogel region removes excess reporters through affinity interactions . . . . This new microfluidic framework provides a unique quantitative assay for NA detection.” (Id., abstract.) FIG. 3 of the instant disclosure, adapted from FIG. 1 of the above article, shows the principle of this technology.

Another article, entitled Isotachophoresis with ionic spacer and two-stage separation for high sensitivity DNA hybridization assay, Eid et al., Analyst, 2013, 138, 3117 (“Eid et al. Analyst 2013”), describes “an on-chip electrophoretic assay for rapid and high sensitivity nucleic acid (NA) detection. The assay uses isotachophoresis (ITP) to enhance NA hybridization and an ionic spacer molecule to subsequently separate reaction products. In the first stage, the probe and target focus and mix rapidly in free solution under ITP. The reaction mixture then enters a region containing a sieving matrix, which allows the spacer ion to overtake and separate the slower probe-target complex from free, unhybridized probes. This results in the formation of two focused ITP peaks corresponding to probe and probe-target complex signals.” (Id., Abstract.)

The above-described on-chip ITP technique achieves ultra-rapid reaction, but it only achieves moderate sensitivity and the specificity of detection is low.

Deoxyribozymes, or DNAzymes, are catalytic DNAs, i.e. DNAs that have catalytic functions. DNAzymes have been used for DNA detection. Catalytic molecular beacons are one type of DNAzyme. FIG. 4 of the instant disclosure, adapted from Locked TASC Probes for Homogeneous Sensing of Nucleic Acids and Imaging of Fixed E. Coli Cells, Sando S, Narita A, Sasaki T, Aoyama Y., Org. Biomol. Chem. 2005; 3:1002-1007, schematically illustrates the principle of DNA detection using catalytic molecular beacons. As shown in FIG. 4, the catalytic molecular beacon in this example has the following modules arranged sequentially from a first end to a second end: a first self-hybridize module (self-hybridize module A), a first target arm (target arm A), a segment containing a cleaved module, a second self-hybridize module (self-hybridize module B), a segment containing a catalytic core, and a second target arm (target arm B). A fluorescent tag F is attached to the self-hybridize module A at the first end, and a quencher Q is attached to the end of the self-hybridize module B that is closer to the second end. In an inactive form of the catalytic molecular beacon, when it is not bound to a target DNA sequence, the self-hybridize module A and self-hybridize module B hybridize with each other, so that the fluorescent tag F and the quencher Q are located in close spatial proximity to each other, and the fluorescence of the fluorescent tag F is quenched. In the presence of a target DNA, the catalytic molecular beacon binds to the DNA and changes to an active form, in which the target arm A and target arm B hybridize to the target DNA in a manner that the cleaved module and the catalytic core are located in close spatial proximity to each other so that the catalytic core cleaves the cleaved module. After cleavage, when the two cleaved fragments are released from the target DNA, the fluorescent tag F is no longer quenched by the quencher Q and will emit fluorescence.

Other catalytic molecular beacons are described in Catalytic Molecular Beacons, Stojanovic M N, de Prada P, Landry D W. ChemBioChem. 2001; 2:411-415; and DNAzyme Amplification of Molecular Beacon Signal, Tian Y, Mao C. Talanta. 2005; 67:532-537. Binary DNAzymes are another type of DNAzymes that can be used for DNA detection.

One type of binary DNAzyme, MNAzyme (Multicomponent Nucleic Acid enzyme), is described in MNAzymes, a Versatile New Class of Nucleic Acid Enzymes That Can Function as Biosensors and Molecular Switches, Mokany et al. JACS 2010; 132(3):1051-9, Jan. 27, 2010 (“Mokany et al. JACS 2010”). FIG. 5 of the instant disclosure, adapted from FIG. 1 of the above article, schematically illustrates the principle of DNA detection using MNAzyme. The MNAzyme is formed of two oligonucleotide components or subunits, part A and part B (referred to as partzymes A and B in the above article). Each part has a target arm, a substrate arm, and a sequence in between that is a part of a catalytic core (partial catalytic core). Initially in an inactive state where the two parts are separate, the binding of the target arms of parts A and B to the target DNA (referred to as the assembly facilitator in the above article) forms the active MNAzyme structure, which can then hybridize to the MNAzyme substrate sequence and catalyze its cleavage. More specifically, the substrate arms of the two parts of the active MNAzyme structure hybridize to the MNAzyme substrate sequence, and the two partial catalytic cores form a catalytic core to cleave the bound substrate. The substrate (also referred to as probe) has a fluorescent tag at one end and a quencher at another end, and the fluorescence of the fluorescent tag is quenched by the quencher due to their close spatial proximity; after cleavage and when the two cleaved fragments are released, the fluorescent tag and the quencher are on different fragments, so the fluorescent tag is no longer quenched and will emit fluorescence. Because the catalysis is carried out by the active MNAzyme structure with multiple turnovers, the MNAzyme introduces an amplification effect into the DNA detection. A method for using MNAzymes for DNA detection is also described in U.S. Pat. No. 8,394,946.

Other types of binary DNAzymes are described in A Binary Deoxyribozyme for Nucleic Acid Analysis, Kolpashchikov D M. ChemBioChem. 2007; 8:2039-2042; and RNA-Cleaving Deoxyribozyme Sensor for Nucleic Acid Analysis: The Limit of Detection, Gerasimova Y V, Cornett E, Kolpashchikov D M, ChemBioChem. 2010; 12: 11 (6):811-729.

The DNAzyme technique can provide signal amplification (i.e. high sensitivity) and high specificity, but the reaction speed is slow.

### SUMMARY

There is a need for not only highly-sensitive but also rapid and inexpensive DNA detection techniques.

As outlined above, on-chip isotachophoresis (ITP) technology enables ultra-rapid nucleic acid detection technology, and DNAzyme technology provides amplified nucleic acid detection signal. Embodiments of the present invention combine ITP and DNAzyme technologies in a technology, referred to herein as the DNAzyme-ITP technology, to conduct DNAzyme reaction using on-chip isotachophoresis. This technology can realize ultra-rapid, highly-sensitive and specific DNA detection. This disclosure describes how to achieve high signal-to-noise ratio in the DNAzyme-ITP technology.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve these and/or other objects, as embodied and broadly described, the present invention provides a DNA detection method which includes: providing a microfluidic chip having a first reservoir containing a low-mobility trailing electrolyte (TE), a second reservoir containing a high mobility leading electrolyte (LE), and a fluid channel between the first and second reservoirs; mixing a sample containing a target DNA sequence or no target DNA sequence and a deoxyribozyme (DNAzyme) sensor, wherein the DNAzyme sensor is either (1) a catalytic molecular beacon which includes a fluorescent tag, the catalytic molecular beacon being capable of cleaving itself in the presence of the target DNA sequence to generate a fluorescent fragment, or (2) a binary DNAzyme and a probe, the binary DNAzyme being capable of cleaving the probe in the presence of the target DNA sequence to generate a fluorescent fragment; loading the mixture of the sample and the DNAzyme sensor into the first reservoir of the microfluidic chip; applying a voltage between the first and second reservoirs; and detecting a fluorescent signal in a detection region of the fluid channel located between the first and second reservoirs. In another aspect, the present invention provides a DNA detection method which includes: providing a microfluidic chip having a first reservoir containing a low-mobility trailing electrolyte (TE), a second reservoir containing a high mobility leading electrolyte (LE), and a fluid channel between the first and second reservoirs, wherein the fluid channel includes a separation region; mixing a sample containing a target DNA sequence or no target DNA sequence and a deoxyribozyme (DNAzyme) sensor, wherein the DNAzyme sensor is either (1) a catalytic molecular beacon which includes a fluorescent tag, the catalytic molecular beacon being capable of cleaving itself in the presence of the target DNA sequence to generate a fluorescent fragment capable of passing through the separation region, the intact catalytic molecular beacon being incapable of passing through the separation region, or (2) a binary DNAzyme and a probe, the binary DNAzyme being capable of cleaving the probe in the presence of the target DNA sequence to generate a fluorescent fragment capable of passing through the separation region, the intact probe being incapable of passing through the separation region; loading the mixture of the sample and the DNAzyme sensor into the first reservoir of the microfluidic chip; applying a voltage between the first and second reservoirs; and detecting a fluorescent signal in a detection region of the fluid channel located between the second reservoir and the separation region.

It is to be understood that both the foregoing general description and the following detailed description are exemplary and explanatory and are intended to provide further explanation of the invention as claimed.

### BRIEF DESCRIPTION OF THE DRAWINGS

FIGS. 1, 2A and 2B illustrate the principle of DNA detection using ITP and a setup for the on-chip ITP assay in an existing method.

FIG. 3 illustrates the principle of a known DNA detection method using ITP and a capture gel.

FIG. 4 shows the principle of a known DNA detection method using catalytic molecular beacons.

FIG. 5 shows the principle of a known DNA detection method using MNAzyme.

FIGS. 6A and 6B schematically illustrate a method of DNA detection using DNAzymes and ITP according to a first embodiment of the present invention.

FIGS. 7A and 7B schematically illustrate a method of DNA detection using DNAzymes and ITP according to a second embodiment of the present invention.

FIGS. 8A to 9B schematically illustrate a method of DNA detection using DNAzymes and ITP according to a third embodiment of the present invention.

### DETAILED DESCRIPTION OF PREFERRED EMBODIMENTS

Embodiments of the present invention provide a novel combination of on-chip isotachophoresis (ITP) and DNAzyme technologies for DNA detection, referred to herein as the DNAzyme-ITP technology. Depending on how the signal is extracted, there are two categories of methods: (I) extracting the signal from the site where the DNAzyme reaction is taking place (simultaneous signal extraction), and (II) extracting the signal from a separated site where the DNAzyme reaction is not taking place (separated signal extraction). Simultaneous signal extraction methods (I) can be done using a relatively simple setup as compared to the separated signal extraction method (II). On the other hand, in terms of signal to noise ratio, the separated signal extraction method (II) is preferable.

Two types of DNAzyme sensors may be used in embodiments of this invention, which respectively contain either (1) a catalytic molecular beacon or (2) a binary DNAzyme and a fluorescent modified probe which can be cleaved by the binary DNAzyme.

In a simultaneous signal extraction method (I) according to some embodiments, the sample is loaded to a microfluidic chip, and the fluorescent signal of the catalytic molecular beacon or the probe is detected at the same location of the chip where the DNAzyme reaction takes place. However, the signal to noise ratio for this technique tends to be relatively low due to significant background signal associated with quenching inefficiency. In the separated signal extraction method (II) according to alternative embodiments, the microfluidic chip is provided with a separation region that separates cleaved and un-cleaved catalytic molecular beacons or probes so that the fluorescent signal is extracted from a separated site where the DNAzyme reaction is not taking place. These alternative methods provide improved signal-to-noise ratio, but the microfluidic chip required is relatively more complex.

The basic process of the DNAzyme-ITP technology according to various embodiments of the present invention is as follows:

First, obtain either (1) the catalytic molecular beacon or (2) the binary DNAzyme and the probe. Then, mix the sample containing target DNA sequences with either (1) the catalytic molecular beacon or (2) the binary DNAzyme and the probe, and load the mixture onto the microfluidic chip. DNAzyme reaction will proceed in the isotachophoresis condition in an ultra-rapid manner, and the fluorescent signal from the cleaved catalytic molecular beacon or probes is detected. Specific embodiments are described in more detail below.

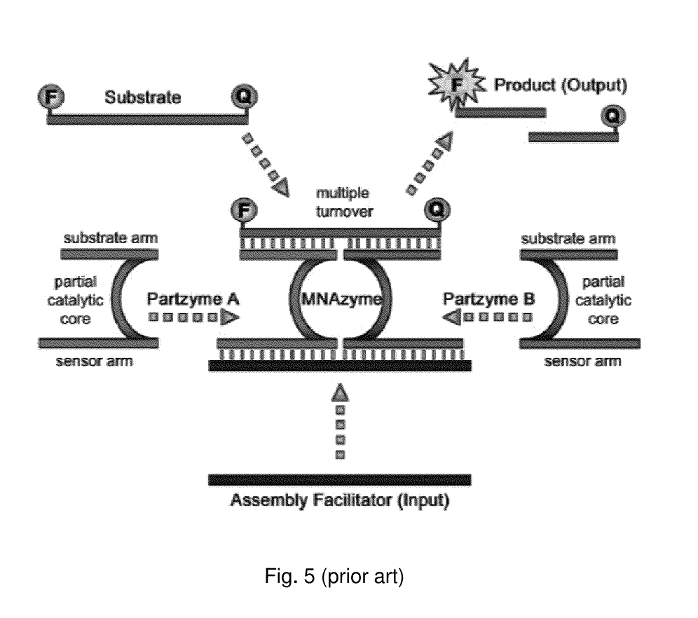

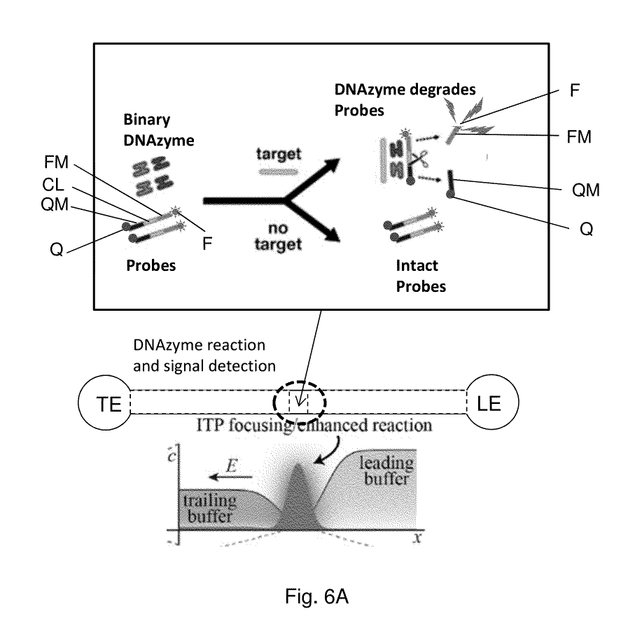

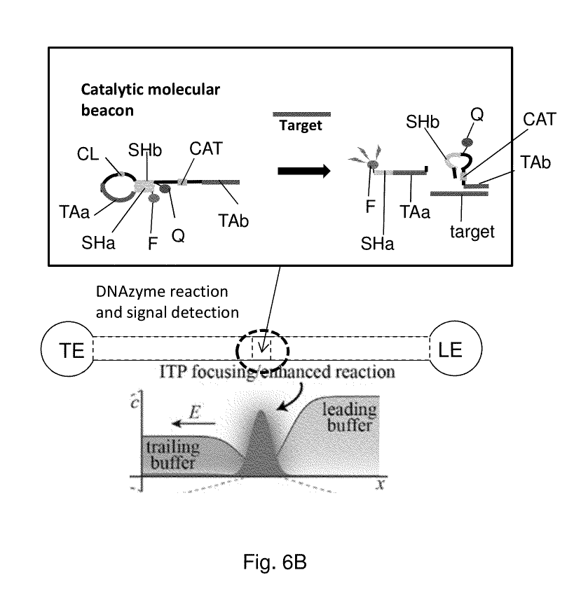

A first embodiment, shown in FIG. 6A, uses a binary DNAzyme and a probe, with simultaneous signal extraction. The binary DNAzyme (similar to the one shown in FIG. 5) has two parts; each part includes a target arm, a substrate arm, and a partial catalytic core. Initially in an inactive state where the two parts are separate, the binding of the respective target arms of the two parts to the target DNA forms the active binary DNAzyme structure, which can then hybridize to the probe and catalyze its cleavage. A FRET (Förster resonance energy transfer or fluorescence resonance energy transfer) quenching probe is used. The probe has three modules (see FIG. 6A): a quencher module QM modified by a quencher Q, a cleaved module CL capable of being cleaved by the binary DNAzyme activated by target sequences, and a fluorescent module FM modified by a fluorescent dye F. The cleaved module is located between the quencher module and the fluorescent module. The probe is capable of hybridizing to and being cleaved by the activated binary DNAzyme and emits fluorescent signal when cleaved. Un-cleaved probe does not emit fluorescent signal due to quenching.

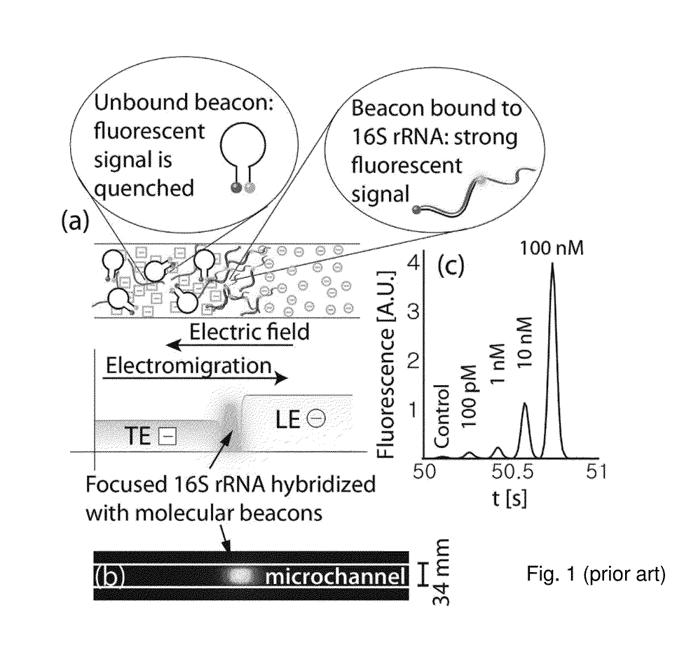

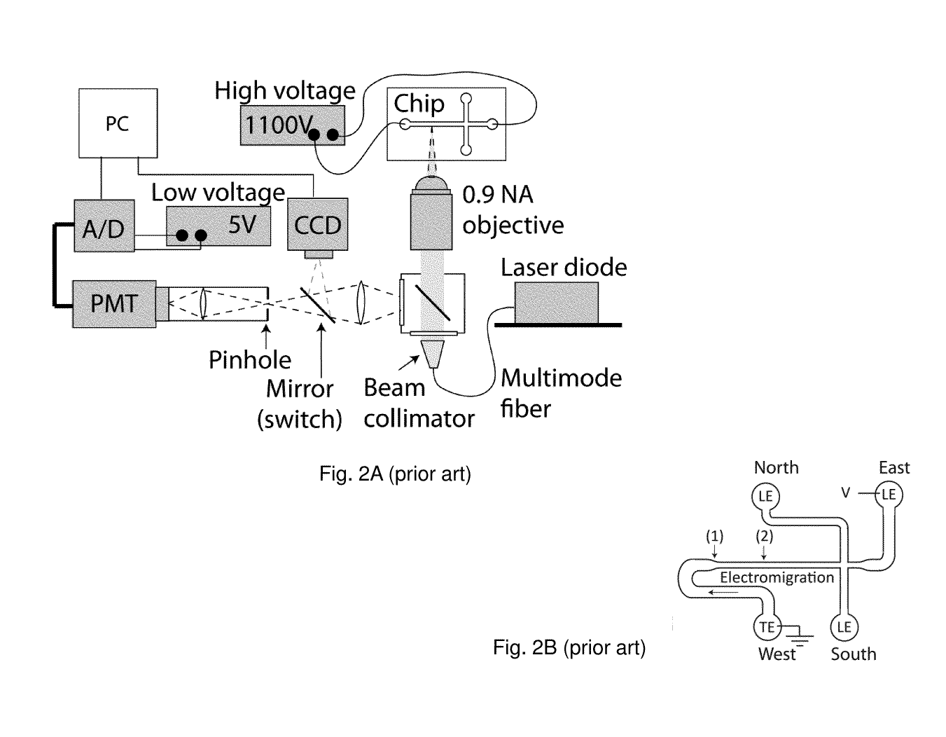

A sample containing target DNA sequences (or no target DNA sequence), the binary DNAzyme subunits, and the probe are mixed. The mixture is assayed using an on-chip ITP setup such as that described in Bercovici et al. Analytical Chemistry 2011 (see FIGS. 1 and 2). The mixture is loaded into a TE reservoir of the microfluidic chip; the binary DNAzyme is activated by the target DNA sequences and DNAzyme reaction takes place in the isotachophoresis condition. The probes cleaved by the binary DNAzyme emit a fluorescent signal after cleavage and the fluorescent signal is detected on site where the DNAzyme reaction is taking place. Of course, if the sample does not contain the target DNA sequence, then the binary DNAzyme is not activated, and the probe is not cleaved and does not emit a fluorescent signal.

The DNAzyme-ITP technology can achieve high sensitivity, high specificity and ultra-rapid reaction. In this first embodiment, the sensitivity can be expected to be about 170 fM, as the sensitivity of the ITP technology described in Bercovici et al. Analytical Chemistry 2011 is about 100 pM and the DNAzyme technology can provide a 600 times sensitivity increase. The reaction time can be expected to be about 10 min.

A second implementation of the first embodiment which uses a catalytic molecular beacon with simultaneous signal extraction is illustrated in FIG. 6B. The catalytic molecular beacon has a fluorescent tag F, a first self-hybridize module SHa, a first target arm TAa, a cleaved module CL, a second self-hybridize module SHb, a quencher Q, a catalytic core CAT, and a second target arm TAb. In the absence of the target DNA, the catalytic molecular beacon is in an inactive form in which the two self-hybridize modules hybridize with each other and the fluorescent tag is located in close spatial proximity of the quencher and is quenched. In the presence of the target DNA, the two target arms hybridize with the target DNA and the catalytic molecular beacon changes to an active form, in which the catalytic core is located in close spatial proximity of the cleaved module and cleaves it. The fluorescent tag and the quencher, which are located on two different sides of the cleaved module, become separated and the fluorescence is no longer quenched.

In this implementation, a sample containing target DNA sequences (or no target DNA sequence) and the catalytic molecular beacons are mixed. The mixture is assayed using an on-chip ITP setup, shown in FIG. 6B, in a procedure similar to that shown and described in FIG. 6A. The mixture is loaded into a TE reservoir of the microfluidic chip; the catalytic molecular beacons are activated by the target sequences and DNAzyme reaction takes place in the isotachophoresis condition. The catalytic molecular beacons are cleaved and emit a fluorescent signal after cleavage, and the fluorescent signal is detected on site where the DNAzyme reaction is taking place. Of course, if the sample does not contain the target DNA sequence, then the catalytic molecular beacon is not activated and not cleaved, and does not emit a fluorescent signal.

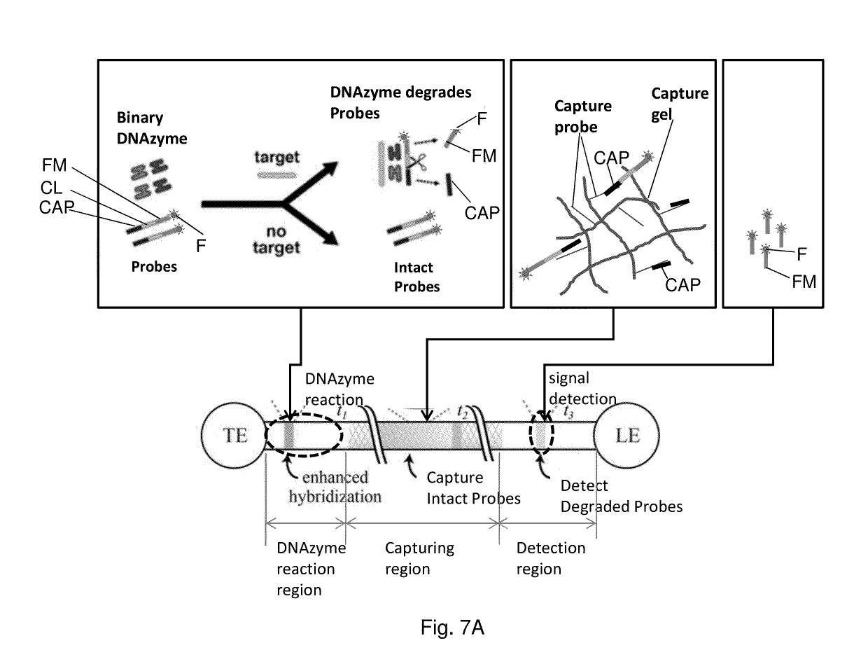

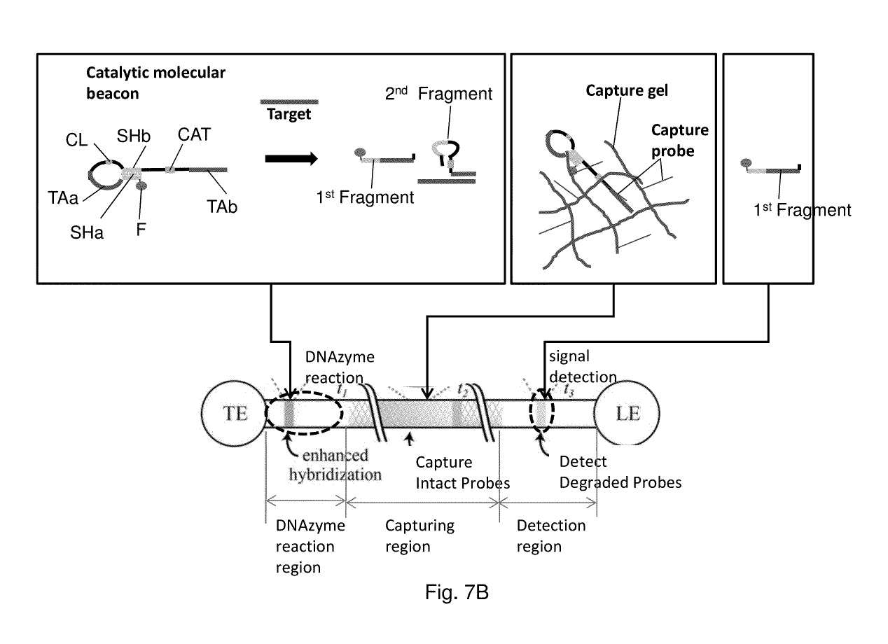

A second embodiment, shown in FIG. 7A, uses a binary DNAzyme and a fluorescent probe with a probe capturing gel to accomplish separated signal extraction, where the location of signal extraction (detection) is separate from the location of the DNAzyme reaction. As shown in FIG. 7A, the probe has three modules: a capture module CAP capable of being captured by the capture gel, a cleaved module CL capable of being cleaved by the binary DNAzyme activated by target sequences, and a fluorescent module FM modified by a fluorescent dye F. The cleaved module is located between the capture module and the fluorescent module. The probe is capable of hybridizing to and being cleaved by the activated binary DNAzyme.

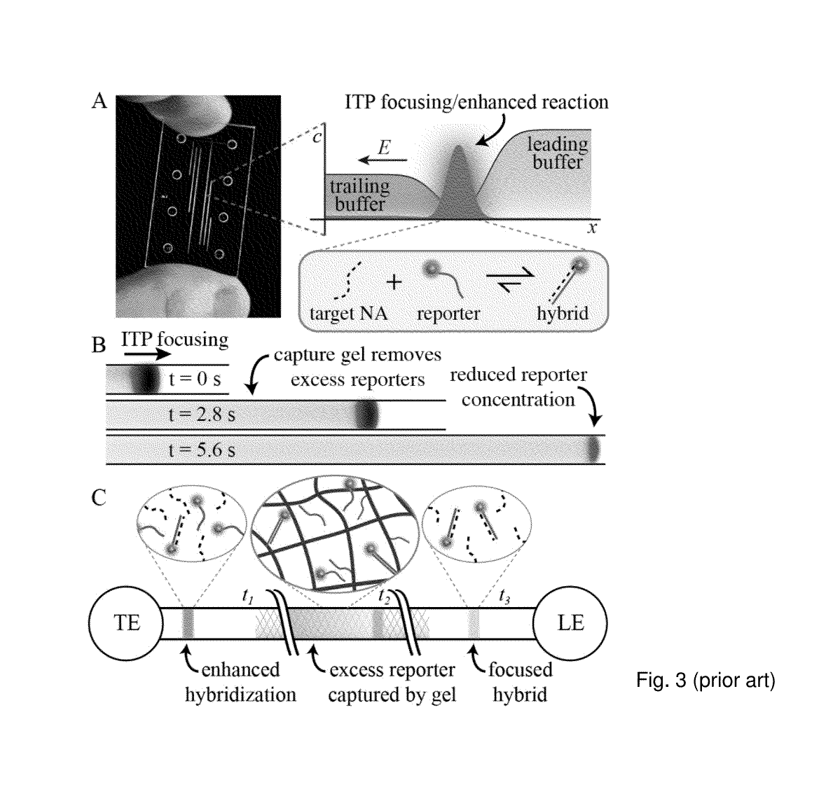

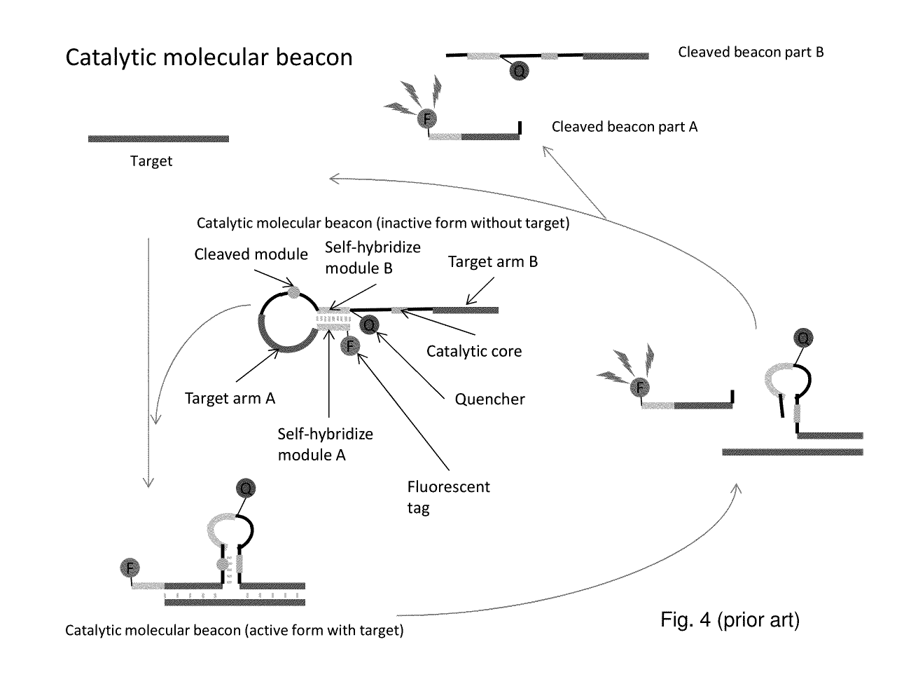

A sample containing target DNA sequences (or no target DNA sequence), the two DNAzyme subunits, and the probes are mixed, and the mixture is assayed using an on-chip ITP setup similar to that described in Garcia-Schwarz et al. Analytical Chemistry 2012 (see FIG. 3). Using this setup, DNAzyme reaction takes place in the isotachophoresis condition, and the un-cleaved probes are captured by the capture gel in a capture region on the microfluidic chip.

As shown in FIG. 7A, the mixture is loaded into a TE reservoir of the microfluidic chip, and enhanced hybridization occurs in a DNAzyme reaction region near the TE reservoir and the probes are cleaved. The ITP zone then migrates through the capture region, into a detection region, where the fluorescence signal is detected.

In the setup described in the Garcia-Schwarz et al. Analytical Chemistry 2012 article, the on-chip capture gel is a DNA-functionalized hydrogel used to remove excess reporters. In the instant embodiment, the capture probe of the capture gel and the capture module of the probe are designed to have complimentary sequences for capturing the capture module of the probe by the capture gel. As a result, intact probes (with their fluorescent modules) and the capture modules of cleaved probes are captured by the capture gel, while the fluorescent modules of the cleaved probes migrate downstream to the detection region where its fluorescence signal is detected. Of course, if the sample does not contain the target DNA sequence, then the binary DNAzyme is not activated and the probes are not cleaved, and no free fluorescent modules will migrate to the detection region.

In this embodiment, the temperatures at the DNAzyme reaction region and the capture region of the microfluidic chip are independently controlled to achieve desired temperatures for the different regions, to optimize both the DNAzyme reaction and capture of the intact probe. For example, a desired temperature for DNAzyme reaction is about 50 degrees C., and a desired temperature for the capture region is typically lower. Temperature is controlled by microfluidic heating method such as heater, Joule heating, Microwave heating etc.

The physical length and/or width of the different regions of the microfluidic chip are optimized respectively to ensure that the mixture will not enter into the capture region while DNAzyme reaction is still taking place, and to ensure that un-cleaved probes will be fully captured in the capture region. This will ensures that when the target DNA sequence is present in the sample, the fluorescent signal in the detection region will be strong because DNAzyme reaction proceeds fully to cleave the probes, and that when the target DNA sequence is not present in the sample, the un-cleaved probes will be captured in the capture regions to minimize erroneous fluorescent signals being detected in the detection region. Preferably, the capture region of the chip is longer and/or wider than the DNAzyme reaction region.

To achieve the above two goals (full DNAzyme reaction when target DNA is present and full capture of un-cleaved probes when target DNA is absent), the voltage applied to the chip may be changed based on the sample position. In the capture region of the chip, the voltage may be decreased or the direction may be changed in order to ensure enough time for capture. More specifically, the sample position can be predicted based on the size of the microchip and the initial voltage; or the sample position can be monitored by monitoring the fluorescent signal in various parts of the chip. At sometime after the sample enters into the capture region (either based on prediction or monitoring), the applied voltage is decreased to reduce moving speed of the sample. The voltage may be increased again after the sample leaves the capture region. Another approach is to change the direction of the applied voltage when the sample is in the capture region. Voltage control may also be done by adding a third electrode in the middle of the chip.

Generally, the length/width of the chip and the voltage applied are designed so that the sample retention time in the capture region is longer than that in the DNAzyme reaction region.

In this second embodiment, the sensitivity can be expected to be about 1.7 fM, as the sensitivity of the ITP technology described in Garcia-Schwarz et al. Analytical Chemistry 2012 is about 1 pM and the DNAzyme technology can provide a 600 times sensitivity increase. The reaction time can be expected to be about 10 min.

A second implementation of the second embodiment which uses a catalytic molecular beacon with a capturing gel to accomplish separated signal extraction is illustrated in FIG. 7B. The catalytic molecular beacon in this embodiment is similar to that used in the second implementation of the first embodiment (FIG. 6B), except that the quencher is not present. In the presence of the target DNA, the catalytic molecular beacon changes from an inactive form, in which the two self-hybridize modules hybridize with each other, to an active form, in which the two target arms hybridize with the target DNA and the catalytic core cleaves the cleaved module.

In this implementation, a sample containing target DNA sequences (or no target DNA sequence) and the catalytic molecular beacons are mixed. The mixture is assayed using an on-chip ITP setup, shown in FIG. 7B, in a procedure similar to that shown and described in FIG. 7A. The mixture is loaded into a TE reservoir of the microfluidic chip, and enhanced hybridization occurs in a DNAzyme reaction region near the TE reservoir, and the catalytic molecular beacon is cleaved. The cleaved catalytic molecular beacon forms two segments: the first fragment contains the fluorescent tag F, the first self-hybridize module SHa, and the first target arm TAa, and the second fragment contains the second self-hybridize module SHb, the catalytic core CAT, and the second target arm TAb. The ITP zone then migrates through the capture region, into a detection region, where the fluorescence signal is detected.

In this implementation, the capture probe of the capture gel is designed to have a complimentary sequence of the second target arm TAb or another part of the second fragment of the catalytic molecular beacon. A specifically designed capture sequence may be added to the catalytic molecular beacon at the second end (i.e. added to the end of the second target arm TAb). As a result, intact catalytic molecular beacons (with their fluorescent tags), as well as second fragments of the cleaved catalytic molecular beacons containing the second target arm TAb, are captured by the capture gel. The first fragments of the cleaved catalytic molecular beacons, which contains the fluorescent tags, migrate downstream to the detection region and their fluorescence signal is detected. Of course, if the sample does not contain the target DNA sequence, then the catalytic molecular beacon is not activated and not cleaved, and no first cleaved fragments will migrate to the detection region.

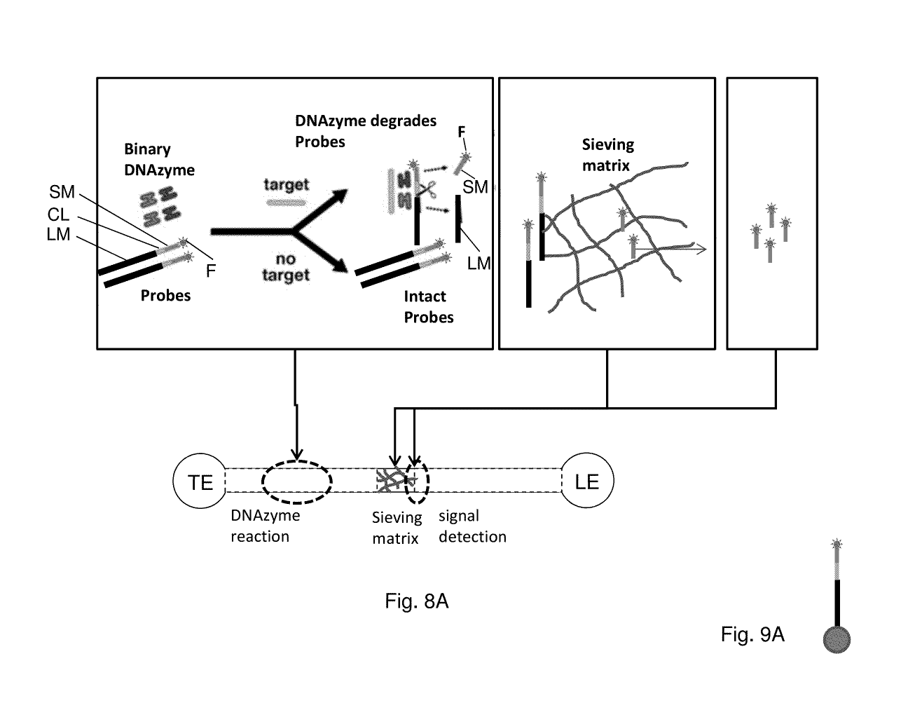

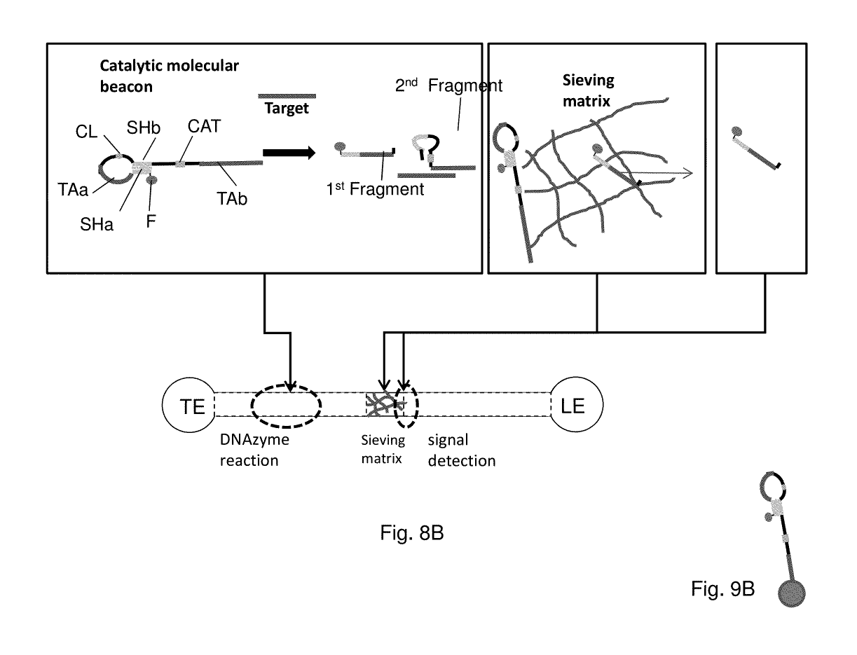

A third embodiment, shown in FIG. 8A, uses a binary DNAzyme and a fluorescent probe with a sieving matrix to accomplish separated signal extraction. The probe has three modules: a long module LM, a cleaved module CL capable of being cleaved by the binary DNAzyme activated by target sequences, and a short module SM modified by a fluorescent dye F. The cleaved module is located between the long module and the short module. The size difference between the intact probe and the short module is large enough to enable them to be separated by the sieving matrix in the microfluidic chip. The probe is capable of hybridizing to and being cleaved by the activated binary DNAzyme.

A sample containing target DNA sequences (or no target DNA sequence), the two DNAzyme subunits, and the probe are mixed, and the mixture is assayed using an on-chip ITP setup similar to that described in Eid et al. Analyst 2013. Using this setup, DNAzyme reaction takes place in the isotachophoresis condition, and the un-cleaved probes are trapped by the sieving matrix on the chip.

As shown in FIG. 8A, the mixture is loaded into a TE reservoir of the microfluidic chip, and enhanced hybridization occurs in a DNAzyme reaction region near the TE reservoir. The ITP zone then migrates through the sieving matrix, into a detection region, where the fluorescence signal is detected.

The sieving matrix separates the probes based on size. Intact probes (with their fluorescent tags) and the long modules of cleaved probes are trapped by the sieving matrix, while the short modules of the cleaved probes with their fluorescent tags are not trapped and can migrate downstream through the sieving matrix to the detection region, where their fluorescence signal is detected. In order to separate intact probe and cleaved short modules sufficiently, it is preferable to modify the long module terminus with a molecule(s) which has physically non-linear bulky shape such as circle, triangle, rectangle, hairpin, spiral etc. (see FIG. 9A). Molecule(s) which are bigger than the sieving matrix pore size are more preferable. The setup described in the Eid et al. Analyst 2013 article uses a sieving matrix to separate single and double stranded DNA. In the instant embodiment, an appropriate sieving matrix is designed to separate the intact probe and the free short module of the cleaved probe.

In this third embodiment, the sensitivity can be expected to be about 0.4 fM, as the sensitivity of the ITP technology described in Eid et al. Analyst 2013 is about 220 fM and the DNAzyme technology can provide a 600 times sensitivity increase. The reaction time can be expected to be about 10 min.

A second implementation of the third embodiment which uses a catalytic molecular beacon with a sieving matrix to accomplish separated signal extraction is illustrated in FIG. 8B. The catalytic molecular beacon in this embodiment is similar to that used in the second implementation of the second embodiment (FIG. 7B), except that the second target arm TAb is longer, or a long tail is attached to the second target arm at the second end of the catalytic molecular beacon. The catalytic molecular beacon is activated by the target DNA in a manner similar to the catalytic molecular beacon in the second implementation of the first and second embodiment.

In this implementation, a sample containing target DNA sequences (or no target DNA sequence) and the catalytic molecular beacons are mixed. The mixture is assayed using an on-chip ITP setup, shown in FIG. 8B, in a procedure similar to that shown and described in FIG. 8A. The mixture is loaded into a TE reservoir of the microfluidic chip, and enhanced hybridization occurs in a DNAzyme reaction region near the TE reservoir, and the catalytic molecular beacon is cleaved. The cleaved catalytic molecular beacon forms two segments: the first (short) fragment contains the fluorescent tag F, the first self-hybridize module SHa, and the first target arm TAa, and the second (longer) fragment contains the second self-hybridize module SHb, the catalytic core CAT, and the second target arm TAb (which is long). The ITP zone then migrates through the sieving matrix, into a detection region, where the fluorescence signal is detected.

The sieving matrix separates the catalytic molecular beacons and its cleaved fragments based on size. Intact catalytic molecular beacons (with their fluorescent tags) and the second (long) fragments of cleaved catalytic molecular beacons are trapped by the sieving matrix, while the first (short) fragments of the cleaved catalytic molecular beacons are not trapped and can migrate downstream through the sieving matrix to the detection region, where their fluorescence signal is detected. In order to separate the intact catalytic molecular beacons and the cleaved short fragments sufficiently, it is preferable to modify the terminus of the long fragments with molecule(s) which has physically non-linear bulky shape such as circle, triangle, rectangle, hairpin, spiral etc. (see FIG. 9B). The molecule(s) which is bigger than the sieving matrix pore size are more preferable. The setup described in the Eid et al. Analyst 2013 article uses a sieving matrix to separate single and double stranded DNA. In the instant embodiment, an appropriate sieving matrix is designed to separate the intact catalytic molecular beacons and the free short segments of the cleaved catalytic molecular beacons.

Comparing to the second or third embodiments, the first embodiment has higher noises or lowed signal to noise ratio, the noise being generated by un-cleaved probe due to insufficient quenching. For this reason, separated signal extraction is more preferable as it can separate the un-cleaved probe (noise source) from the cleaved probe. However, the first embodiment has a simpler setup.

It is noted that parts of the illustrations in FIGS. 6A, 7A and 8A here are adopted from, with modifications, the Garcia-Schwarz et al. Analytical Chemistry 2012 article and an article entitled A Plasmonic DNAzyme Strategy for Point-of-Care Genetic Detection of Infectious Pathogens, Kyryl Zagorovsky, Dr. Warren C. W. Chan, first published online: 10 Feb. 2013, the abstract of which is available at http://onlinelibrary.wiley.com/doi/10.1002/anie.201208715/abstract:jsessionid=9F16112AEE9CC6AA1823B9C25E0C293B.f04t01.

In each of the above embodiments, two implementations are described, respectively using (1) a catalytic molecular beacon and (2) a binary DNAzyme and a fluorescent modified probe as the DNAzyme sensor. Binary DNAzymes have better target specificity than catalytic molecular beacons, while catalytic molecular beacons have somewhat better sensitivity. Both have high sensitivity.

The DNA detection methods described in this disclosure can realize high-sensitive, specific, and ultra-rapid DNA detection using a simple setup.

It will be apparent to those skilled in the art that various modification and variations can be made in the DNAzyme-ITP DNA detection method and related apparatus of the present invention without departing from the spirit or scope of the invention. Thus, it is intended that the present invention cover modifications and variations that come within the scope of the appended claims and their equivalents.

What is claimed is:

1. A DNA detection method comprising:
providing a microfluidic chip having a first reservoir containing a low-mobility trailing electrolyte (TE), a second reservoir containing a high mobility leading electrolyte (LE), and a fluid channel between the first and second reservoirs;
loading a sample containing a target DNA sequence or no target DNA sequence and a deoxyribozyme (DNAzyme) sensor into the first reservoir of the microfluidic chip, wherein the DNAzyme sensor is either (1) a catalytic molecular beacon which includes a fluorescent tag, the catalytic molecular beacon being capable of cleaving itself in the presence of the target DNA sequence to generate a fluorescent fragment, or (2) a binary DNAzyme and a probe, the binary DNAzyme being capable of cleaving the probe in the presence of the target DNA sequence to generate a fluorescent fragment;
applying a voltage between the first and second reservoirs; and
detecting a fluorescent signal in a detection region of the fluid channel located between the first and second reservoirs.

2. The method of claim 1, wherein the DNAzyme sensor is the catalytic molecular beacon, which includes a fluorescent tag, a first self-hybridize module, a first target arm, a cleaved module, a second self-hybridize module, a quencher, a catalytic core, and a second target arm, wherein the catalytic molecular beacon is capable of changing from an inactive form to an active form upon hybridizing to the target DNA sequence, wherein in the inactive form the first and second self-hybridize modules hybridize with each other and the fluorescent tag is located in close spatial proximity of the quencher and is quenched, wherein in the active form the two target arms hybridize with the target DNA sequence and the catalytic core is located in close spatial proximity of the cleaved module to cleaves it, wherein the fluorescent fragment resulting from the cleavage includes the fluorescent tag, the first self-hybridize module and the first target arm.

3. The method of claim 1, wherein the DNAzyme sensor is the binary DNAzyme and the probe, wherein the binary DNAzyme includes two subunits, each subunit including a target arm, a substrate arm, and a partial catalytic core, wherein the binary DNAzyme is capable of changing from an inactive form to an active form upon hybridizing to the target DNA sequence, wherein in the active form the target arms of the two subunits hybridize to the target DNA sequence, the substrate arms of the two subunits are capable of hybridizing to the probe, and the two partial catalytic cores of the two subunits form a catalytic core capable of cleaving the hybridized probe, and
wherein the probe includes a quencher module modified by a quencher, a fluorescent module modified by a fluorescent tag, and a cleaved module located between the quencher module and the fluorescent module, wherein the probe is capable of being cleaved by the active form of the binary DNAzyme, and wherein the fluorescent fragment resulting from the cleavage includes the fluorescent module modified by the fluorescent tag.

4. A DNA detection method comprising:
providing a microfluidic chip having a first reservoir containing a low-mobility trailing electrolyte (TE), a second reservoir containing a high mobility leading electrolyte (LE), and a fluid channel between the first and second reservoirs, wherein the fluid channel includes a separation region;
loading a sample containing a target DNA sequence or no target DNA sequence and a deoxyribozyme (DNAzyme) sensor into the first reservoir of the microfluidic chip, wherein the DNAzyme sensor is either (1) a catalytic molecular beacon which includes a fluorescent tag, the catalytic molecular beacon being capable of cleaving itself in the presence of the target DNA sequence to generate a fluorescent fragment capable of passing through the separation region, the intact catalytic molecular beacon being incapable of passing through the separation region, or (2) a binary DNAzyme and a probe, the binary DNAzyme being capable of cleaving the probe in the presence of the target DNA sequence to generate a fluorescent fragment capable of passing through the separation region, the intact probe being incapable of passing through the separation region;
applying a voltage between the first and second reservoirs; and
detecting a fluorescent signal in a detection region of the fluid channel located between the second reservoir and the separation region.

5. The method of claim 4, wherein the separation region contains a capture gel which is functionalized with a DNA sequence.

6. The method of claim 5, wherein the DNAzyme sensor is the catalytic molecular beacon, which includes a fluorescent tag, a first self-hybridize module, a first target arm, a cleaved module, a second self-hybridize module, a catalytic core, and a second target arm, wherein the catalytic molecular beacon is capable of changing from an inactive form to an active form upon hybridizing to the target DNA sequence, wherein in the inactive form the first and second self-hybridize modules hybridize with each other, wherein in the active form the two target arms hybridize with the target DNA sequence and the catalytic core is located in close spatial proximity of the cleaved module to cleaves it, wherein the fluorescent fragment resulting from the cleavage includes the fluorescent tag, the first self-hybridize module and the first target arm, and
wherein the DNA sequence of the capture gel of the separation region hybridizes to the second target arm.

7. The method of claim 5, wherein the DNAzyme sensor is the binary DNAzyme and the probe, wherein the binary DNAzyme includes two subunits, each subunit including a target arm, a substrate arm, and a partial catalytic core, wherein the binary DNAzyme is capable of changing from an inactive form to an active form upon hybridizing to the target DNA sequence, wherein in the active form the target arms of the two subunits hybridize to the target DNA sequence, the substrate arms of the two subunits are capable of hybridizing to the probe, and the two partial catalytic cores of the two subunits form a catalytic core capable of cleaving the hybridized probe,
wherein the probe includes a capture module, a fluorescent module modified by a fluorescent tag, and a cleaved module located between the capture module and the fluorescent module, wherein the probe is capable of being cleaved by the active form of the binary DNAzyme, and wherein the fluorescent fragment resulting from the cleavage includes the fluorescent module modified by the fluorescent tag, and
wherein the DNA sequence of the capture gel of the separation region hybridizes to the capture module.

8. The method of claim 4, wherein the separation region contains a sieving matrix.

9. The method of claim 8, wherein the DNAzyme sensor is the catalytic molecular beacon, which includes a fluorescent tag, a first self-hybridize module, a first target arm, a cleaved module, a second self-hybridize module, a catalytic core, and a second target arm, wherein the catalytic molecular beacon is capable of changing from an inactive form to an active form upon hybridizing to the target DNA sequence, wherein in the inactive form the first and second self-hybridize modules hybridize with each other, wherein in the active form the two target arms hybridize with the target DNA sequence and the catalytic core is located in close spatial proximity of the cleaved module to cleaves it, wherein the fluorescent fragment resulting from the cleavage includes the fluorescent tag, the first self-hybridize module and the first target arm, and
wherein the fluorescent fragment is capable of passing through the sieving matrix of the separation region and the intact catalytic molecular beacon is incapable of passing through the sieving matrix.

10. The method of claim 9, wherein the second target arm contains a group with a non-linear bulky shape.

11. The method of claim 8, wherein the DNAzyme sensor is the binary DNAzyme and the probe, wherein the binary DNAzyme includes two subunits, each subunit including a target arm, a substrate arm, and a partial catalytic core, wherein the binary DNAzyme is capable of changing from an inactive form to an active form upon hybridizing to the target DNA sequence, wherein in the active form the target arms of the two subunits hybridize to the target DNA sequence, the substrate arms of the two subunits are capable of hybridizing to the probe, and the two partial catalytic cores of the two subunits form a catalytic core capable of cleaving the hybridized probe,
wherein the probe includes a capture module, a fluorescent module modified by a fluorescent tag, and a cleaved module located between the capture module and the fluorescent module, wherein the probe is capable of being cleaved by the active form of the binary DNAzyme, and wherein the fluorescent fragment resulting from the cleavage includes the fluorescent module modified by the fluorescent tag, and
wherein the fluorescent fragment is capable of passing through the sieving matrix of the separation region and the intact probe is incapable of passing through the sieving matrix.

12. The method of claim 11, wherein the capture module contains a group with a non-linear bulky shape.
